(19) United States
(12) Patent Application Publication
(10) Pub. No.: US 2006/0009424 A1
(43) Pub. Date: Jan. 12, 2006

(54) Nasaleaze

(76) Inventor: Anatoly Kogan, Los Angeles, CA (US)

Correspondence Address: ANTOLY KOGAN, 833 SOUTH BEDFORD STREET, APT 105, LOS ANGELES, CA, 90035, US

(21) Appl. No.: 10/710,382

(22) Filed: Jul. 6, 2004

(51) Int. Cl. A61K 31/60

(52) U.S. Cl. 514/165

(57) ABSTRACT

Applicant has an idea that the healing powers of combining available “over the counter” medicines Neomycin, Polymyxin B Sulfates and Bacitracin Zinc first aid antibiotic ointment, and Hidrocortizone anti itch ointment produces a medicine named “Nazalease”. When inserted into the nasal cavity, “Nazalease” is capable of treating unwanted nasal deposits. The proposed medication named “Nasaleaze” consists of 50% of Neomycin, Polymyxin B Sulfates and Bacitracin Zinc including inactive ingredients as described in U.S. Pat. No. 5,652,274 and White Petrolatum or any other generic medication consisting of the same parameters and 50% of 1% Hidrocortizone and White Petrolatum or any other generic medication consisting of the same parameters. The composition applied in amounts equaling the size of q-tip head to the inside of the nostrils and spread around by gently squeezing nostrils. “Nasaleaze” proposed use: by adults and children 2 years of age and older.

Nasal congestion healing composition and method for preparation and use.

“A method of the treatment of the hardened mucus deposits of nasal cavities comprising to a subject in need of such treatment a composition of 1% of Hydrocortisone 10, present in the amount of 50% of the total composition and Original Neosporin Ointment, that contains in itself as main ingredients Neomycin, Polymyxin and Bacitracin Zinc, comprising another 50% of the total composition wherein a therapeutically effective amount of the composition is applied in the nasal cavity of subject”.

1. The idea of mixing together medicines available “over the counter” produces a new medication. When being applied into the nasal cavity, it effectively treats unwanted deposits by virtue of it being present on the surface.
